(19) United States
(12) Patent Application Publication
(10) Pub. No.: US 2012/0010460 A1
(43) Pub. Date: Jan. 12, 2012

(54) TISSUE DISPLACING METHOD

(75) Inventors: Seigo KITANO, Beppu-shi (JP); Kazuhiro YASUDA, Oita-shi (JP); Takahiro KOGASAKA, Tokyo (JP); Kunihide KAJI, Tokyo (JP); Ken YAMATANI, Tokyo (JP); Nobuko MATSUO, Tokyo (JP)

(73) Assignee: OLYMPUS MEDICAL SYSTEMS CORP., Tokyo (JP)

(21) Appl. No.: 13/178,927

(22) Filed: Jul. 8, 2011

Related U.S. Application Data
(60) Provisional application No. 61/362,908, filed on Jul. 9, 2010.

(51) Int. Cl. A61B 17/00; A61B 1/005

(52) U.S. Cl. 600/37

(57) ABSTRACT

An organ displacing method implemented for performing a procedure in a patient's body cavity, the method includes a gravitational movement step in which the body position of the patient is changed, whereby surrounding organs adjacent to a hollow organ are moved in a primary direction by gravity; and a non-gravitational movement step in which, by using a medical instrument inserted into the hollow organ, the hollow organ is moved in a secondary direction where the hollow organ is separated from the surrounding organs.

### BACKGROUND OF THE INVENTION

1. Field of the Invention

The present invention relates to a tissue displacing method implemented with respect to an arbitrary tissue in a body cavity during a procedure which is performed by inserting a medical instrument into the body cavity. This application claims priority to and the benefits of Provisional Application No. 61/362, 908 filed on Jul. 9, 2010, the disclosure of which is incorporated herein by reference.

2. Description of Related Art

Heretofore, as an example of minimally invasive treatment, a variety of procedures such as cholecystectomy has been performed by using a laparoscope. During this type of laparoscopic operation, a plurality of holes is opened in the abdominal wall, and a plurality of instruments is inserted thereto.

Recently, in order to minimize invasiveness by further reducing the number of holes opened in the abdominal wall, a procedure performed by inserting a flexible endoscope through a patient's natural orifices such as the mouth, the nose, and the anus has been suggested. As a medical instrument used for this type of procedure, for example, an endoscope for a procedure as disclosed in US Patent Application Publication No. 2007/0249897 has been suggested.

Various organs and tissues neighbor one another in each of the human body cavities, such as the abdominal cavity. When a space necessary for a procedure is not secured since, for example, a target region of the procedure is covered by other organs or tissues, an operator moves the organs or tissues as appropriate by using hand, forceps, or the like. The operator holds the moved tissues or the like by displacing them using the forceps or the like, and secures a space necessary for the procedure.

### SUMMARY OF THE INVENTION

According to a first aspect of the present invention, an organ displacing method, which is implemented for performing a procedure in a patient's body cavity, the method includes a gravitational movement step in which the body position of the patient is changed, whereby surrounding organs adjacent to a hollow organ are moved in a primary direction by gravity; and a non-gravitational movement step in which, by using a medical instrument inserted into the hollow organ, the hollow organ is moved in a secondary direction where the hollow organ is separated from the surrounding organs.

According to a second aspect of the present invention, the medical instrument includes a flexible tube having a curving portion that can curve at the tip thereof.

According to a third aspect of the present invention, the medical instrument is an endoscope which includes the flexible tube.

According to a fourth aspect of the present invention, during the gravitational movement step, a fluid is supplied into the hollow organ so as to dilate the hollow organ before the body position of the patient is changed.

According to a fifth aspect of the present invention, after the gravitational movement step, the fluid is collected, and the dilation of the hollow organ is released.

According to a sixth aspect of the present invention, after the dilation of the hollow organ is released, the non-gravitational movement step is performed.

According to a seventh aspect of the present invention, a procedure region as a target of the procedure is located at the hollow organ.

According to a ninth aspect of the present invention, the hollow organ is the stomach.

According to a tenth aspect of the present invention, the procedure is the Heller-Dor procedure with respect to esophageal achalasia.

### BRIEF DESCRIPTION OF THE DRAWINGS

FIG. 1 is a view illustrating a process of an organ displacing method of an embodiment of the present invention.

FIGS. 2 and 3A are views illustrating a dilation step in the organ displacing method.

FIG. 3B is a view illustrating an image observed by an endoscope in the state of FIG. 3A.

FIG. 4 is a view illustrating a state where the patient's body position has been changed.

FIG. 5 is a view illustrating an operation of releasing the state of gastric dilation.

FIGS. 6 and 7A are views illustrating a non-gravitational movement step in the organ displacing method.

FIG. 7B is a view illustrating an image observed by the endoscope in the state of FIG. 7A.

FIG. 8 is a view illustrating a state where a space for a Heller-Dor procedure has been formed.

FIGS. 9 and 10 are views illustrating a modified example of the dilation step in the organ displacing method.

FIG. 11 is a view illustrating a treatment endoscope which can be used for the organ displacing method of the embodiment of the present invention.

### DETAILED DESCRIPTION OF THE INVENTION

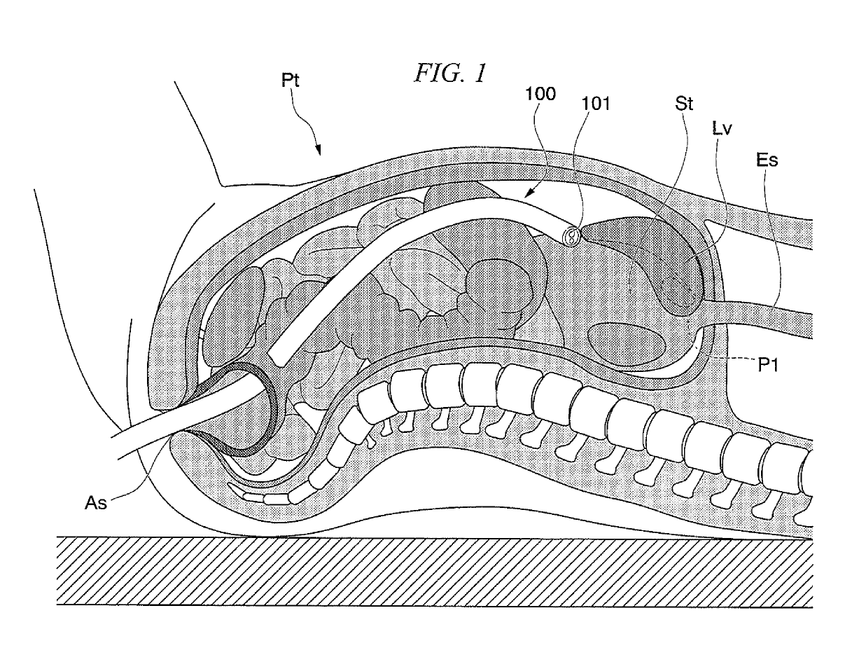

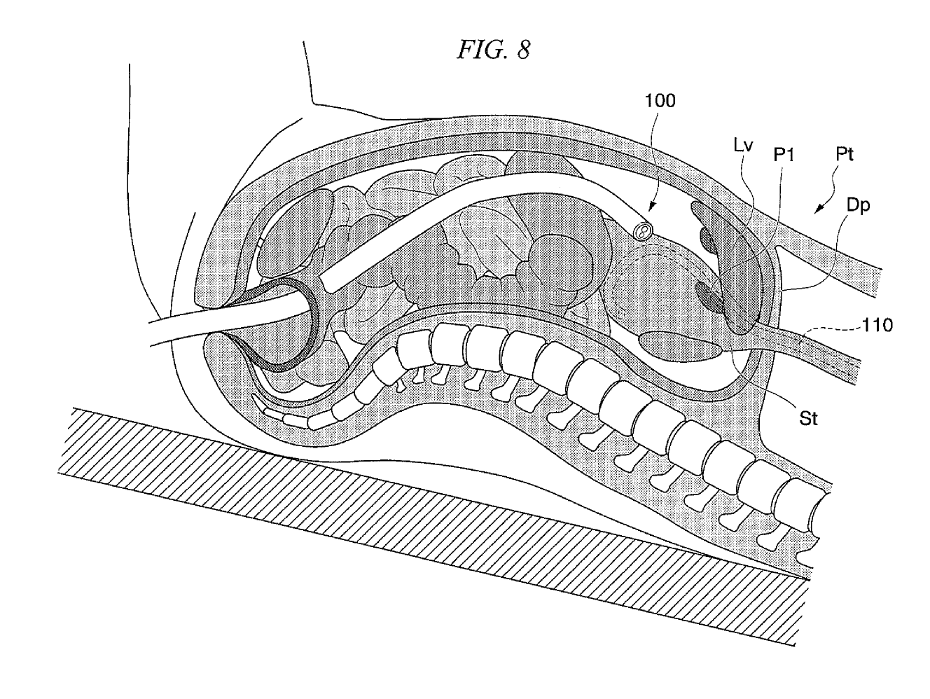

Hereinafter, the procedure method of an embodiment of the present invention will be described with reference to FIGS. 1 to 10.

The organ displacing method of the invention is a method for forming a sufficient space for performing a procedure between a primary tissue where a region subjected to the procedure (procedure region) is located and a secondary tissue which is positioned at the periphery of the primary tissue. The organ displacing method of the invention can be suitably used, for example, in a state where the procedure region is covered by the secondary tissue when an external force is not applied. In the embodiment, a case will be described where the Heller-Dor procedure which is a myotomy with fundoplication with respect to the esophageal achalasia is performed by respectively taking the stomach (a hollow organ) and the liver (a surrounding organ) as the primary and secondary tissues for example.

FIG. 1 is a view illustrating a process of the organ displacing method of the embodiment. A procedure region P1 on which the Heller-Dor procedure is performed is located at an area around the cardia in the gastric fundus which is in the vicinity of the junction of the stomach St and esophagus Es. However, in the supine position in which the Heller-Dor procedure is usually performed, the liver Lv is on the procedure region P1 so as to cover the procedure site, as shown in FIG. 1. Accordingly, it is not easy to secure a sufficient space for performing the Heller-Dor procedure unless the liver Lv is moved and held by displacing or the like.

In the embodiment, in order to perform the Heller-Dor procedure while suppressing invasiveness with respect to the patient, first, the operator inserts an endoscope 100 including an observation device 101 through an anus As which is a natural orifice of a patient Pt and makes an incision in the intestinal wall, thereby introducing the endoscope into the abdominal cavity. Instead of the endoscope 100, for example, an endoscope for a procedure disclosed in US Patent Application, Publication No. 2007/0249897 may be used.

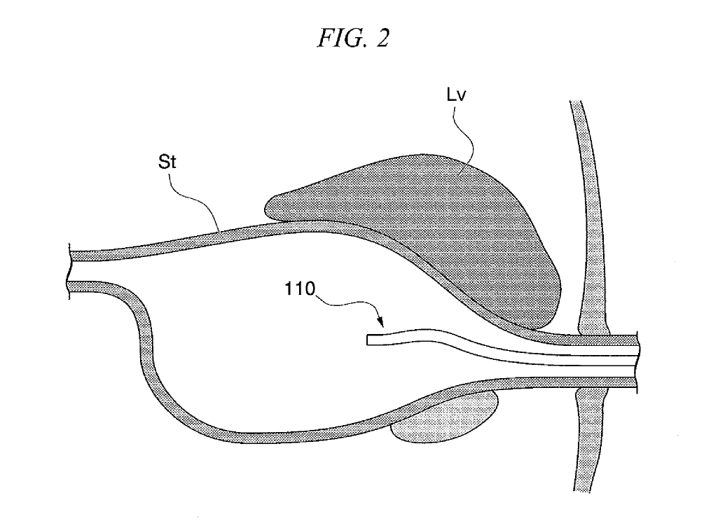

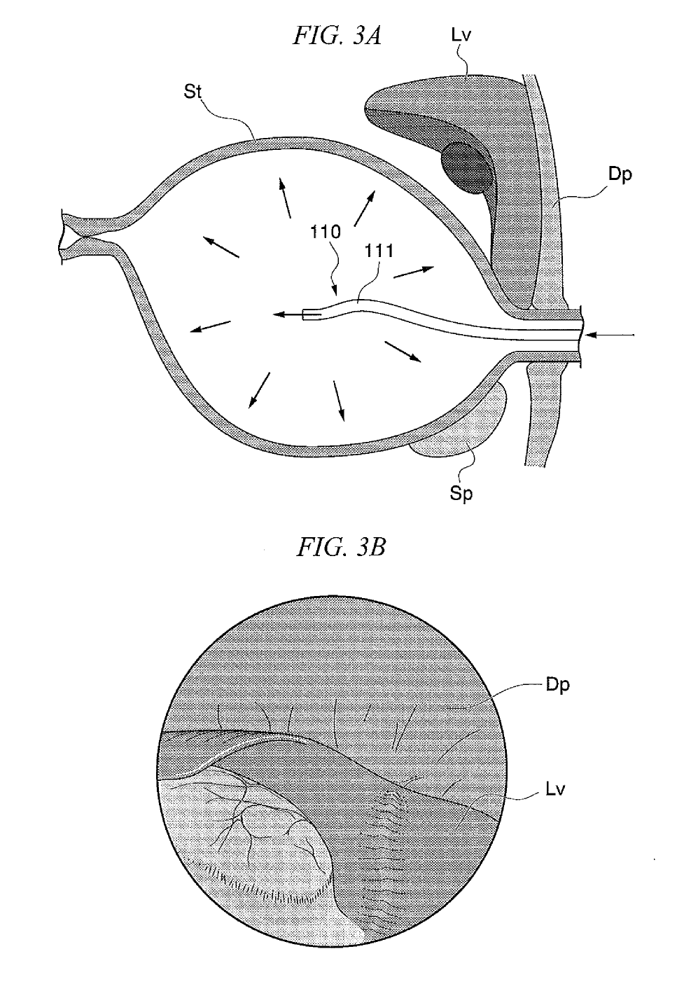

Thereafter, as shown in FIG. 2, the operator introduces another endoscope 110 into the stomach separately St from the endoscope 100, and then feeds a fluid such as carbon dioxide into the stomach through a channel (not shown) of the endoscope 110 (dilation step), as shown in FIG. 3. The tip side of the endoscope 110 is provided with a flexible tube including a curving portion 111 that can curve as an insertion portion. The fluid is supplied in an amount sufficiently larger than the amount of fed air in the usual endoscopic observation, for example, in an amount of about 1 liter, so that the extended gastric wall sufficiently displaces the liver Lv.

By the supply of the fluid, the stomach St dilates to the gastric wall side as a whole. At this time, due to the gastric wall extended by the dilation, the positional relationship between the surrounding organs changes, whereby the liver Lv and the spleen Sp are displaced so as to be separated from the anterior gastric wall.

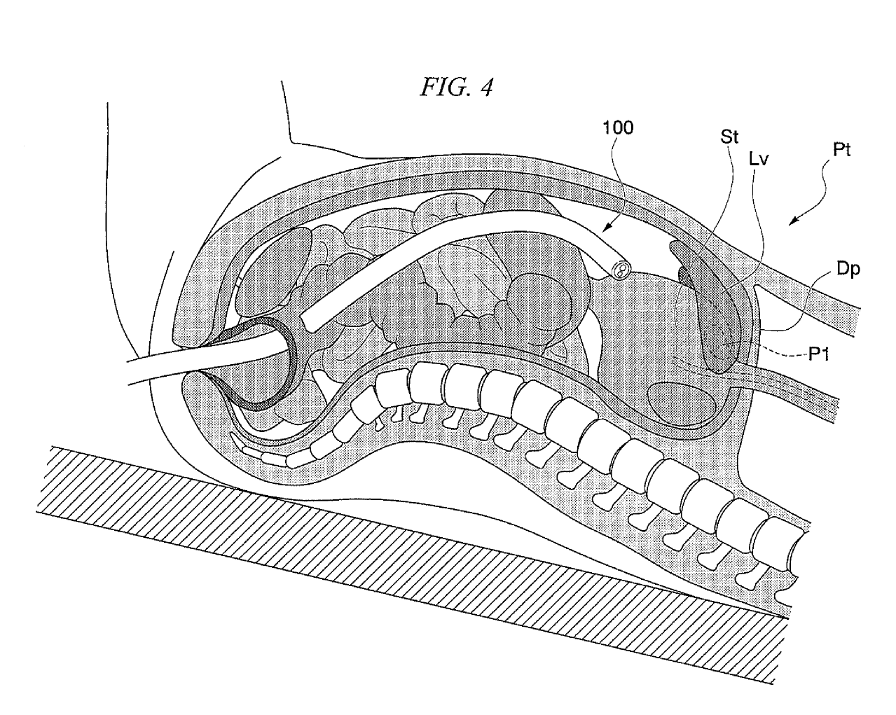

Subsequently, as shown in FIG. 4, the operator changes the body position of the patient Pt to lower the head side. Due to this change in body position, both the stomach St and the liver Lv move in a primary direction in which the stomach St and the liver Lv face the head side by gravity and are pushed against the diaphragm Dp (a gravitational movement step). It is desirable that the inclination angle in the body position change is appropriately adjusted while the state of the liver Lv is observed by the endoscope 100 so that the liver Lv is sufficiently displaced toward the diaphragm Dp. Although the liver Lv is displaced by the gravitational movement step, the stomach St where the procedure region P1 is located is also moved toward the diaphragm Dp by gravity. Therefore, at this point in time, a space for performing the Heller-Dor procedure is not yet formed around the procedure site P1.

In the embodiment, the gravitational movement step may be performed before or after the dilation step. However, if the gravitational movement step is performed in a state where the stomach St has dilated after the dilation step, the stomach St moves toward the diaphragm Dp as if the stomach St rolls over, and the liver Lv is suitably pushed to the diaphragm Dp as shown in the image observed by the endoscope 100 in FIG. 3B, which is thus preferable.

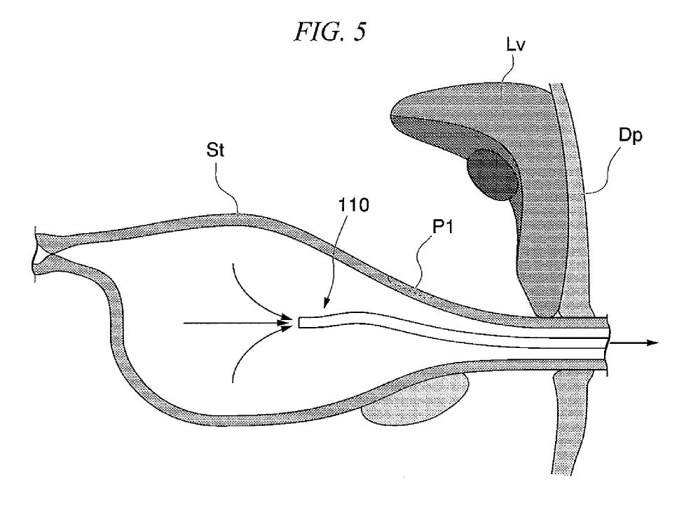

While holding the body position of the patient Pt, the operator collects the fluid supplied into the stomach by using the endoscope 110, as shown in FIG. 5. As a result, the dilation state of the stomach St is released so that the stomach St contracts. As necessary, the body position may be returned to the original horizontal state thereafter.

When the procedure region is located at the posterior gastric wall side as in a case of omentectomy procedure, it is preferable to maintain a state where the fluid has been supplied into the stomach even during the procedure.

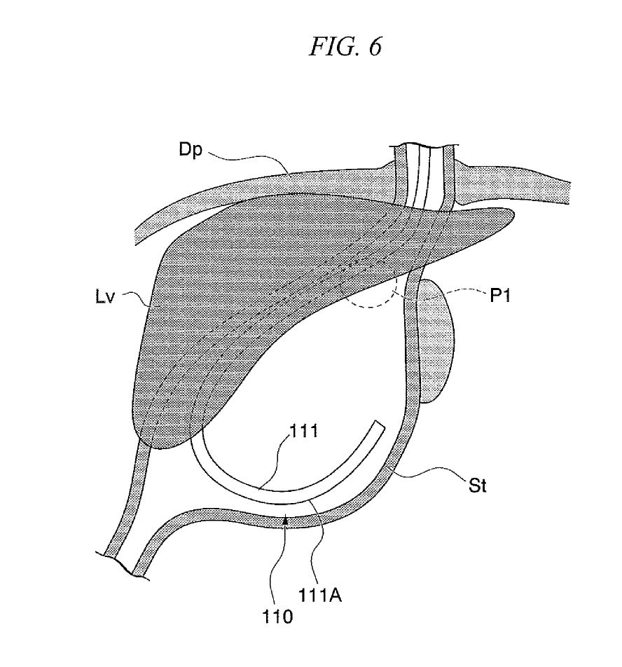

Next, the operator operates the curving portion 111 of the endoscope 110 to curve the curving portion as shown in FIG. 6. During the curving operation, the curving portion 111 is sufficiently curved (preferably, the tip thereof is curved at a curving angle of 90° or more) so that the operation of pushing the stomach which will be described later can be suitably performed.

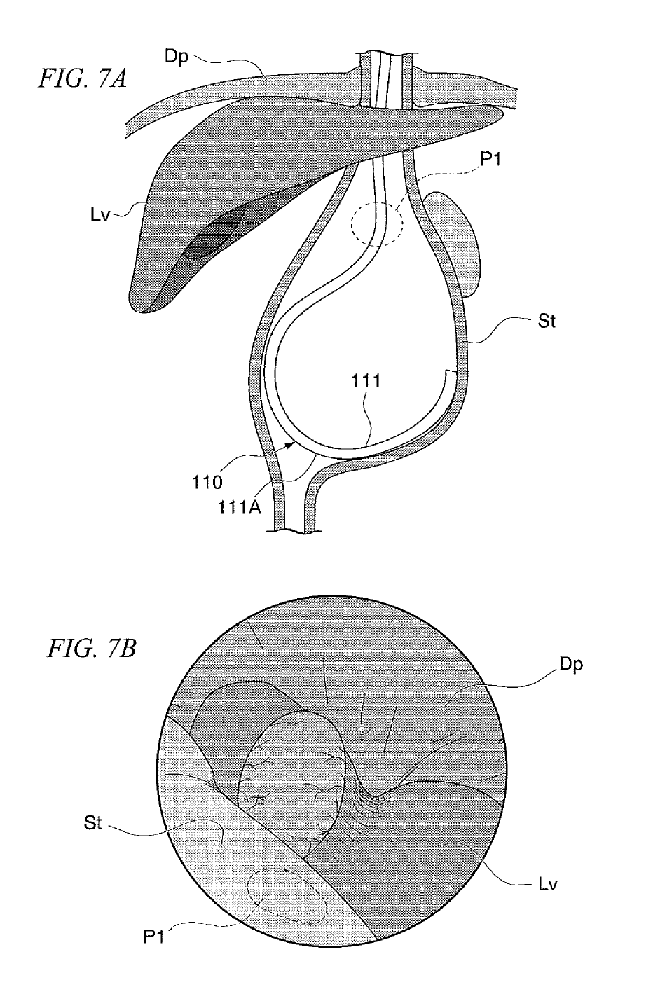

After curving the curving portion 111, the operator pushes the endoscope 110 to push the outside (back-side) 111A of the curvature in the curving portion 111 against the gastric wall, as shown in FIG. 7A. The operator further pushes the endoscope 110 to move the stomach St toward the anus. By this operation, the stomach St moves in the direction (a secondary direction) in which the stomach St is separated from the diaphragm Dp and the liver Lv against the gravity generated by the body position change (non-gravitational movement step), and a space for performing the Heller-Dor procedure on the procedure region P1 is formed between the stomach St and the liver Lv as shown in FIGS. 7B (image observed by the endoscope 100) and 8. At this time, if the anterior wall of the stomach St is flattened by appropriately adjusting the pushing direction and the pushing intensity of the endoscope 110 while the image observed by the endoscope 100 is viewed, it is possible to secure a larger space and to easily perform the myotomy of the Heller-Dor procedure.

While maintaining the state where the endoscope 110 has been pushed, the operator introduces a treatment tool such as a high-frequency knife or the like into the abdominal cavity through the channel of the endoscope 100, and approaches the exposed procedure region P1. Subsequently, while observing the procedure region with the endoscope 100, the operator performs the Heller-Dor procedure by using the treatment tool.

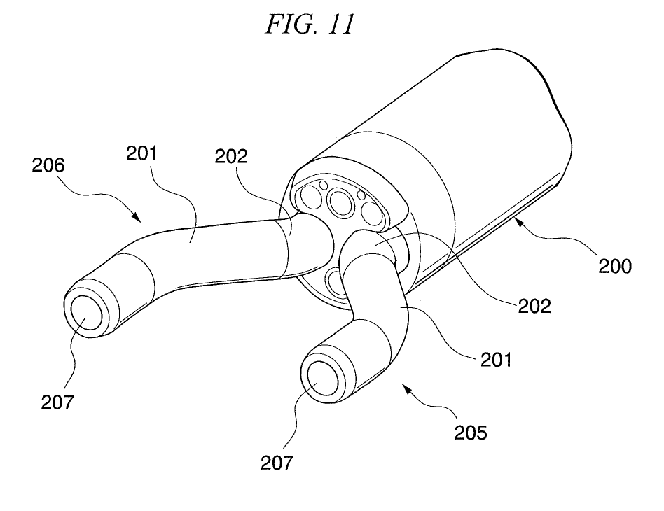

Further, in the preceding embodiment, an example of using the endoscope 100 is explained. However, the present invention is not limited thereto. A treatment endoscope 200 shown in FIG. 11 may be used instead of a viewing endoscope, such as the endoscope 100. The treatment endoscope 200 is provided with two arms 205, 206 at a distal end thereof. Both of the arms 205, 206 have bending portions 201, 202 and a channel for a treatment tool to freely pass through. An aperture 207 communicating with the channel is formed on a distal end of each arm 205, 206. Thereby, the present application can be performed with various treatment tools which protrude from the apertures 207 of the treatment endoscope 200.

The treatment tool such as the high-frequency knife or the like can be simply introduced through the same access path as that of the endoscope 100. However, the device is not necessarily introduced in this manner, but may approach the procedure region P1 through an access path different from that of the endoscope 100. The series of operations described above is performed at both sides including the head side and the anus side of the patient Pt. Accordingly, the operations may be performed as necessary by a plurality of operators or by an operator with an assistant.

According to the organ displacing method of the present embodiment, during the gravitational movement step, the liver Lv as a surrounding organ is displaced toward the diaphragm Dp. In addition, during the non-gravitational movement, while the liver Lv is kept displaced, the stomach St as a hollow organ and the procedure region P1 located at the stomach St are moved in the direction in which the stomach St and the procedure region P1 are separated from the diaphragm Dp and the liver Lv. By these two steps, a space with such a size that the procedure can be suitably performed on the procedure region P1 is formed between the liver Lv and the stomach St.

In the Heller-Dor procedure that has been performed hitherto by means of laparotomy and a laparoscope, in order to form a space necessary for the procedure by exposing the procedure site, the adjacent tissues such as the liver and the spleen have been displaced manually or with forceps. If the Heller-Dor procedure is performed through the natural orifices so as to further minimize the invasiveness, the tissues cannot be displaced manually as described above. Moreover, if the forceps or the like are used for displacement, the following problems are caused. That is, as in the present embodiment, when the hollow organ is as large as the stomach, a substantially strong force is necessary for the organ movement as described above; therefore, high strength and rigidity are required for the forceps, so the forceps increase in size accordingly. Consequently, in order to introduce the forceps or the like to the abdominal cavity, it is necessary to make a small incision in the abdominal wall to install a trocar, thus the effect of the original purpose, minimizing invasiveness, is diminished.

According to the organ displacing method of the present embodiment, by changing the body position of the patient Pt during the gravitational movement step, it is possible to displace the liver Lv without using forceps or the like. Accordingly, during the non-gravitational movement step, by operating the medical instrument inserted through the natural orifices and using the present method in combination with the endoscope 100 or the like, a procedure such as the Heller-Dor procedure which requires organ displacement can also be minimally invasive when performed through a natural orifice.

Furthermore, during the non-gravitational movement step, since the endoscope 110 larger than the general treatment tool is used, even a relatively large tissue such as the stomach St can be moved by generating a sufficient force. Moreover, since the endoscope 110 is curved so as to push and move the stomach St with the back-side 111A of the curvature, the region where the endoscope 110 and the internal gastric wall contact each other becomes a curved surface, and the contact area increases. Accordingly, it is possible to reduce the burden to the gastric tissue and the patient in the stomach movement operation.

In addition, since the dilation step is performed by using functions of feeding air, water and the like which are provided to the general endoscopic device, the step can be performed by means of a usual endoscope without requiring other treatment tool.

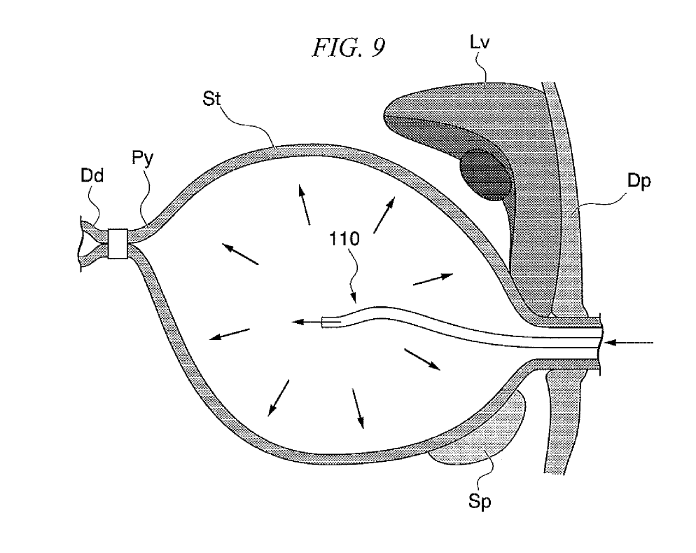

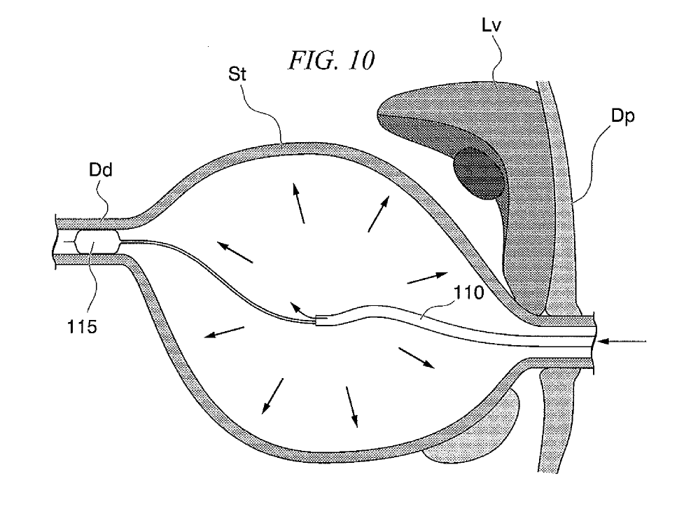

In the embodiment described above, in order to prevent the inflation of the gastrointestinal tract caused by the supplied fluid, carbon dioxide that is easily absorbed into the body is employed as the fluid in the dilation step. However, the inflation of the gastrointestinal tract may be prevented by other methods. For example, as shown in FIG. 9, by ligating the pylorus Py or the duodenum Dd by means of the treatment tool introduced to the abdominal cavity, or, as shown in FIG. 10, by temporarily closing the duodenum Dd by inflating a balloon 115 introduced through the channel of the endoscope 110 within the duodenum Dd, a state where the stomach St can be inflated may be created.

Herein, an embodiment of the present invention has been described, but the invention is not limited thereto. Within a range that does not depart from the scope of the invention, addition, omission, substitution or other modifications of the configuration can be mane.

For example, as the natural orifices through which the medical instrument such as the endoscope 100 is introduced, the vagina or the like can be used in addition to the anus. Furthermore, though not a natural orifice, a hole may be formed by incising the navel in which a scar is not easily noticed, whereby the endoscope 100 or the like may be introduced through this hole. In this case, although the patient will suffer from slight invasiveness, postoperative appearance is minimally affected, and the psychological burden on the patient can be reduced.

In the organ displacing method of the present embodiment, either the gravitational movement step or the non-gravitational movement step may be performed first. Furthermore, contrary to the above described example, the primary tissue may be moved in the gravitational movement step, and then the secondary tissue may be moved and displaced in the non-gravitational movement step. For example, when a procedure is performed on the gallbladder, even if the sequence is almost the same as the above describe operation, the gallbladder becomes the primary tissue, and the stomach becomes the secondary tissue, so the stomach is moved and displaced in the non-gravitational movement step.

In the organ displacing method of the present invention, for example, if it would be better to decrease the degree of displacing the surrounding organ in the content of the procedure, one or two of the steps may be omitted among the dilation step, the gravitational movement step, and the non-gravitational movement step.

What is claimed is:

1. An organ displacing method implemented for performing a procedure in a patient's body cavity, the method comprising:
a gravitational movement step in which the body position of the patient is changed, whereby surrounding organs adjacent to a hollow organ are moved in a primary direction by gravity; and
a non-gravitational movement step in which, by using a medical instrument inserted into the hollow organ, the hollow organ is moved in a secondary direction where the hollow organ is separated from the surrounding organs.

2. The organ displacing method according to claim 1,
wherein the medical instrument includes a flexible tube having a curving portion that can curve at the tip thereof.

3. The organ displacing method according to claim 2,
wherein the medical instrument is an endoscope which includes the flexible tube.

4. The organ displacing method according to claim 1,
wherein, during the gravitational movement step, a fluid is supplied into the hollow organ so as to dilate the hollow organ before the body position of the patient is changed.

5. The organ displacing method according to claim 4,
wherein, after the gravitational movement step, the fluid is collected, and the dilation of the hollow organ is released.

6. The organ displacing method according to claim 5,
wherein, after the dilation of the hollow organ is released, the non-gravitational movement step is performed.

7. The organ displacing method according to claim 1,
wherein a procedure region as a target of the procedure is located at the hollow organ.

8. The organ displacing method according to claim 7,
wherein the hollow organ is the stomach.

9. The organ displacing method according to claim 7,
wherein the procedure is “Fundoplication surgery”.

10. The organ displacing method according to claim 9,
wherein the procedure is the Heller-Dor procedure with respect to esophageal achalasia.
